(19) United States
(12) Patent Application Publication
(10) Pub. No.: US 2026/0236007 A1
(43) Pub. Date: Aug. 13, 2026

(54) THE MOBILE 3D PRINTING OF PHARMACEUTICAL DOSAGE FORMS

(71) Applicant: Board of Regents, the University of Texas System, Austin, TX (US)

(72) Inventors: Mohammed Maniruzzaman, Pflugerville, TX (US); Leela Raghava Jaidev Chakka, Austin, TX (US); Vineet Kulkarni, Austin, TX (US); Faez Alkadi, Austin, TX (US)

(21) Appl. No.: 19/153,818

(22) PCT Filed: Feb. 8, 2024
(86) PCT No.: PCT/US2024/015040

§ 371 Date: Aug. 5, 2025

Related U.S. Application Data
(60) Provisional application No. 63/484,185, filed on Feb. 9, 2023.

(51) Int. Cl. G05B 19/4099; B29C 64/386; B33Y 50/00

(52) U.S. Cl. CPC G05B 19/4099; B29C 64/386; B33Y 50/00; G05B 2219/49007

(57) ABSTRACT

Described herein are techniques for the manufacture of medications that include active pharmaceutical ingredient in a custom way. For example, printing system including a fused deposition modeling (FDM) three-dimensional (3D) printer can be used to generate administerable dosage forms, such as comprising different pharmaceutially loaded carriers or functional or inactive materials. Instructions for a printing system can be generated on the fly and in response to physician instructions, clinical data, patient information, such as by using a machine learning model or artificial intelligence system trained to generate dosage forms. The printing system may include a filament palette configured to splice and fuse a plurality of mixture filaments to form a printing load to generate the dosage forms.

### CROSS-REFERENCE TO RELATED APPLICATIONS

This application claims the benefit of and priority to U.S. Provisional Application No. 63/484,185, filed on Feb. 9, 2023, which is hereby incorporated by reference in its entirety.

### FIELD

The present disclosure relates generally to additive manufacturing technology, and more specifically to the production of various dosage forms using machine learning or artificial intelligence algorithms, portable computing devices, and mid-air 3D printing systems.

### BACKGROUND

Traditional pharmaceutical dosage manufacturing involves different steps of blending, extrusion, pressing, and packaging. Besides the drug, various excipients are added at each step during the manufacturing process of a tablet or pill. The multiple steps involve huge machinery, consuming huge amounts of energy in preparing the pharmaceutical doses. The addition of excipients also increases the complexity of the formulations. The huge manufacturing costs related to the extreme sterile conditions employed for manufacturing pharmaceuticals increase the cost of the product. Advances in pharmaceutical dosage manufacturing are needed.

### SUMMARY

As personalized medication and personalized pharmaceutical dosing is a promising field of research and 3D printing technologies are widely-available in people's daily lives, a manufacturing technology that integrates portable computing devices and 3D printers enables patients to easily obtain prescribed medications.

The present disclosure relates, in part, to personalized medication and pharmaceutical dosage fabricated by hot-melt extrusion and 3D printing technologies. Techniques, methods, and systems are disclosed herein for fabricating different types of medication, pharmaceutical dosage, or pharmaceutical delivery carriers with various configurations.

In an aspect, systems of manufacturing printed products are provided. An example system of this aspect comprises a computing device, a multi-dose filament system, and an additive deposition device. In examples, the computing device is configured to send an instruction including a set of composition data and a set of structure data of a printed product. In examples, the multi-dose filament system is configured to receive the instruction from the computing device and to produce a mixture filament including at least one polymeric material and at least one active pharmaceutical ingredient (API) according to the set of composition data received from the computing device. In examples, the additive deposition device is configured to receive the instruction from the computing device and to manufacture the printed product according to the set of structure data by using a printing load that includes the mixture filament. In some examples, the system is powered by a green energy supply. In some examples, the the green energy supply includes a solar panel and a battery. In some examples, the system further comprises at least one energy monitor that measures energy consumption of the system. In some examples, the system comprises at least one in-line monitor that continuously measures a characteristic of the printed product by using back pressure sensors or optical sensors. In some examples, the system comprises at least one filament palette configured to splice and fuse a plurality of mixture filaments to form a printing load. In some examples, the multi-dose filament system is configured to produce the mixture filament by using a plurality of different polymeric materials and/or a plurality of different APIs.

In another aspect, methods of manufacturing printed products are provided. An example method of this aspect comprises receiving a print request, retrieving a prescription related to the print request, determining a set of health parameters of a patient based on the prescription, generating a set of clinical data from the set of health parameters of the patient, providing the set of clinical data to a machine learning model, the machine learning model having been trained to output a printing profile based on clinical data, and sending the printing profile to an additive deposition device for using in manufacturing a printed product. In examples, the printed product comprises at least one polymeric material and at least one API. In some examples, the print request is received from a patient's mobile communication device. In some examples, the prescription related to the print request is retrieved from a remote data center. In some examples, the set of health parameters of the patient is obtained via at least one wearable sensor. In some examples, the health parameters of the patient include self-reported values. In some examples, the printing profile comprises filament selection information and geometry information of the printed product. In some examples, the geometry information comprises a STL file or other computer-readable file that is supported by a Computer-Aided Design (CAD) software. In some examples, the printing profile comprises splicing information that specifies selection of a plurality of printing filaments for the additive deposition device. In some examples, the printing profile comprises composition information that specifies the respective weight percent of the at least one API and the at least one polymeric material for manufacturing the printed product. In some examples, the method further comprises receiving an approval of the print request from a healthcare provider before sending the printing profile to the additive deposition device. In some examples, the method further comprises manufacturing a printed product based on the printing profile via an additive deposition device or other manufacturing device.

In another aspect, methods of manufacturing printed products are provided. An example method of this aspect comprises receiving a print request, retrieving a prescription related to the print request, determining a set of health parameters of a patient based on the prescription, generating a set of clinical data from the set of health parameters of the patient, providing the set of clinical data to a machine learning model, the machine learning model having been trained to output a printing profile based on clinical data, and sending the printing profile to an additive deposition device for using in manufacturing a printed product. In examples, the printed product comprises at least one polymeric material and at least one API. In some examples, the print request is received from a patient's mobile communication device. In some examples, the prescription related to the print request is retrieved from a remote data center. In some examples, the set of health parameters of the patient is obtained via at least one wearable sensor. In some examples, the health parameters of the patient include self-reported values. In some examples, the printing profile comprises filament selection information and geometry information of the printed product. In some examples, the geometry information comprises a STL file or other computer-readable file that is supported by a Computer-Aided Design (CAD) software. In some examples, the method further comprises receiving an approval of the print request from a healthcare provider before sending the printing profile to the additive deposition device. In some examples, the method further comprises manufacturing a printed product based on the printing profile via an additive deposition device. In some examples, the printing profile comprises splicing information that specifies the selection of a plurality of printing filaments for the additive deposition device. In some examples, the printing profile comprises composition information that specifies the respective weight percent of the at least one API and the at least one polymeric material for manufacturing the printed product.

In another aspect, methods of generating and storing instruction files of printed products are provided. An example method of this aspect comprises generating an instruction file, such as an instruction file that comprises a set of printed product design data and a set of processing condition data; manufacturing, such as by using an additive deposition device, a printed product based at least in part on the generated instruction file; assessing, such as by an assessment engine, optionally using a machine learning model, the quality of the printed product; and storing, such as in a database, the instruction file of the printed product that is assessed as satisfactory. In some examples, the set of printed product design data includes geometry of the printed product. In some examples, the set of processing condition data includes nozzle temperature, nozzle type, or number of nozzle of the additive deposition device. In some examples, when the quality of the printed product is assessed an determined to not be satisfactory, methods of this aspect may include discarding the instruction file or storing the instruction file in association with an indicator that the quality was determined to not be satisfactory.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 is a schematic illustration of manufacturing a printed product according to some examples.

FIG. 2 is a flowchart detailing a process for manufacturing printed products according to some examples.

FIG. 3 is a flowchart detailing a process for constructing a database according to some examples.

FIG. 4A shows a filament palette according to some examples.

FIG. 4B shows a configuration of a filament palette and an additive deposition device according to some examples.

FIG. 5A is a schematic illustration of various printed product structures according to some examples.

FIG. 5B shows various printed products according to some examples.

FIG. 6 provides data showing differential scanning calorimetry analysis results of pharmaceutical ingredients, filaments, and printed products according to some examples.

FIG. 7 provides data showing powder X-ray diffraction analysis results of pharmaceutical ingredients, filaments, and printed products according to some examples.

FIG. 8 provides data showing powder X-ray diffraction analysis results of pharmaceutical ingredients, filaments, and printed products according to some examples.

FIG. 9 provides data showing Fourier-transform infrared spectroscopy analysis results of pharmaceutical ingredients, filaments, and printed products according to some examples.

FIG. 10 provides data showing Fourier-transform infrared spectroscopy analysis results of pharmaceutical ingredients, filaments, and printed products according to some examples.

FIG. 11 provides data showing drug release profiles of exemplary pharmaceutical formulations according to some examples.

FIG. 12 provides data showing drug release profiles of exemplary pharmaceutical formulations according to some examples.

FIG. 13 is a schematic illustration of manufacturing filaments and pharmaceutical products according to some examples.

### DETAILED DESCRIPTION

The method and technology disclosed herein may be used in the manufacture of medications that include active pharmaceutical ingredients and excipients, such as polymers, plasticizers, inorganic carriers, etc. The disclosed techniques provide for customized preparation of medications, for example using a system that can compile multiple pharmaceutically loaded filaments into a single printing load. The printing load can then be printed, for example using a fused deposition modeling (FDM) three-dimensional (3D) printer, into administerable dosage forms, such as tablets, films, or the like.

The disclosed techniques can allow for preparation of custom forms, which can have the pharmaceutical dosage, form, size, excipient, or the like varied, such as according to instructions received from a remote system. In some examples, such a remote system can take as input instructions from a physician, who may have access to clinicial data about a patient prescribed the dosage forms. Optionally, a machine learning model or artificial intelligence system can generate instructions for a printing system to prepare the custom dosage forms, which may use as input prescription information, clinical data, patient information, or the like to determine appropriate forms, loadings, and/or compositions for the custom dosage forms. In this way, a printing system can collect data and generate custom dosage forms for a patient, such as in response to a request for a prescription refill generated by the patient, and taking into account various sources of information to allow a customized prescription refill to be generated on the fly.

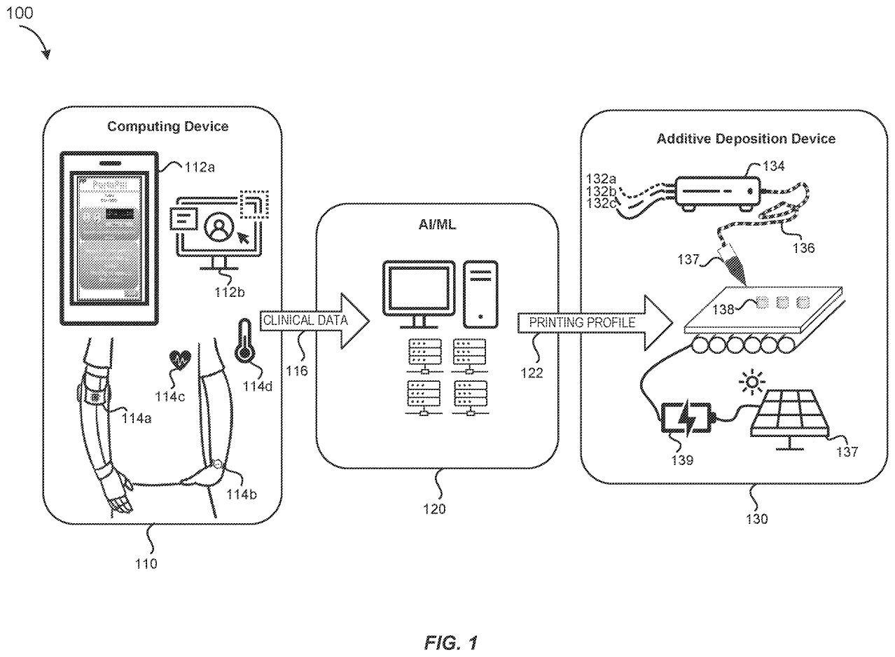

FIG. 1 is a schematic illustration of a system 100 for manufacturing a printed product according to some examples. System 100 includes a computing device 110 that is configured to collect a set of clinical data 116, and employ an artificial intelligence (AI) or machine learning (ML) algorithm 120 that is configured to receive the set of clinical data 116 and to generate an instruction including a printing profile 122 based at least in part on the set of clinical data 116. System 100 also includes an additive deposition device 130 that is configured to receive the instruction and to manufacture a printed product according to the instruction. In some examples, the system 100 may include one or more processors and one or more memory units configured to or capable of storing instructions that, upon execution by the one or more processors, configure or cause the system to perform steps in compliance with the instruction.

The computing device 110 collects and sends the clinical data 116 to the artificial intelligence (AI) or machine learning (ML) algorithm 120 for further processing. In some examples, the computing device 110 may be a mobile communication device. A mobile communication device may be a device that can be easily transported and has remote communication capabilities. Examples of remote communication capabilities include exchanging data between devices over short ranges (e.g., using a Bluetooth standard). Other examples of remote communication capabilities include using a mobile phone (wireless) network, wireless data network (e.g. 4G, 5G, or similar networks), Wi-Fi, Wi-Max, or any other communication medium that may provide access to a network such as the Internet or a private network. Examples of mobile communication devices include mobile phones (e.g. cellular phones), key fobs, PDAs, tablet computers, net books, laptop computers, personal music players, hand-held specialized readers, etc. Further examples of mobile communication devices include wearable devices, such as smart watches, fitness bands, ankle bracelets, rings, earrings, etc., as well as automobiles with remote communication capabilities. In some examples, the computing device 110 may include at least one graphical user interface (GUI) 112a and/or 112b to receive an input (e.g., click, tap, or the like) from a user. The input may indicate that the user selects an item on the screen (e.g., a prescription request, a report of health status, or the like). In yet another example, the device 110 may receive multiple words via keyboard input as a string of natural language. Any suitable input mechanism may be used to perform embodiments and examples of the present disclosure.

The clinical data 116 may include any information, parameter, and/or measurement as needed. The clinical data may be obtained in a real-time manner or accessed from an external source (e.g., a database).

In some examples, the computing device 110 may further include at least one monitoring sensor 114 to collect or measure a set of health parameters of a user or patient to form the clinical data 116. The number or type of the monitoring sensor 114 may be determined and adjusted as needed. The set of health parameters may be stored in a separate data store or database that is communicatively connected to the computing device 110. In some examples, the computing device 110 may include a blood pressure monitor 114a that measures the patient's blood pressure in a continuous or periodic manner. The computing device 110 may also include a glucose monitor 114b that checks the patient's blood sugar levels automatically at timed intervals. The computing device 110 may additionally include a Holter monitor 114c that records the patient's heart rhythm and determines the risk of irregular heartbeats (arrhythmias). The computing device 110 may also include a thermometer 114d to measure the patient's body temperature. Moreover, in some examples, the patient may voluntarily add or provide health parameters as needed through the GUI 112a and/or 112b. In some examples, the computing device 110 may access any health parameter from an external data source as needed. For example, with the patient's permission, the computing system may access and collect the patient's Carbohydrate antigen 19-9 (CA19-9) data from the patient's record retained by the patient's health provider, combine the CA19-9 data with the set of health parameters measured by the at least one monitoring sensor 114, and form the clinical data 116. In some examples, the computing device 110 determines the set of health parameters to be collected or obtained based at least in part on the user's prescription (e.g., medical prescriptions).

In some examples, the artificial intelligence (AI) or machine learning (ML) algorithm 120 is communicatively connected to the computing device 110 to receive and process the clinical data 116. In some examples, the artificial intelligence (AI) or machine learning (ML) algorithm 120 may constitute a module or component of the computing device 110. In some examples, the artificial intelligence (AI) or machine learning (ML) algorithm 120 may be stored or executed on a remote server. Based at least in part on the clinical data 116 and any medical prescription that may be associated with the patient, the artificial intelligence (AI) or machine learning (ML) algorithm 120 can determine and output a printing profile 122. The printing profile 122 may include any information that is relevant to any property of a printed product 138. In some examples, the artificial intelligence (AI) or machine learning (ML) algorithm 120 can access a database containing standardized printing profiles and interpolate between or alter the standardized profiles to accommodate a particular characteristic or clinical data of a patient in order to generate the printing profile.

The printing profile 122 may include, but is not limited to, filament selection and/or combination information, geometry information of the printed product, splicing information that specifies the arrangement of a plurality of printing filaments for the additive deposition device 130, composition information or composition data (e.g., respective weight percentages of multiple active pharmaceutical ingredient (“API”) components and polymeric material component for manufacturing the printed product), API dosage information, structure data of the printed product, or the like. In some examples, the polymeric material comprises a biocompatible or digestible thermoplastic. Exemplary polymeric materials include, but are not limited to, hydroxpropylmethyl cellulose, hydroxypropyl cellulose, or hydroxpropylmethyl cellulose. In some examples, the API is in a gel state or a liquid state. In some examples, the API is in a crystalline state. In some examples, the API is in a semi-crystalline state or an amorphous state. In some specific examples, the at least one API is Nifedipine, Aspirin, chloroquine diphosphate, or Ibuprofen. It will be appreciated that many other APIs may be used according to the disclosed techniques and that reference to these specific APIs is merely to provide some illustrative examples and is not limiting. In some examples, the printing profile may include 3D design file of the printed product as a computer-aided design (CAD) file. In some examples, the 3D design file is a STL file that is supported by a software. In some examples, the printing profile may be included in an instruction that is executable by a computing device or a manufacturing device (e.g., G-code). In some examples, before sending the printing profile 122 to the additive deposition device 130, an approval from the patient's healthcare provider (e.g., electronic approval by the patient's family doctor) may be obtained. In some examples, when the clinical data 116 contains a medical prescription, the patient's health provider may review the clinical data 116 of the patient and adjust the medical prescription as needed, for example to increase or decrease an amount of an API, to select or change a carrier, to select a size or form of the product, to change the prescription to a different API, or the like. Based on the adjusted medical prescription, the artificial intelligence (AI) or machine learning (ML) algorithm 120 may update or regenerate the printing profile 122 accordingly.

In some examples, the additive deposition device 130 is communicatively connected to the artificial intelligence (AI) or machine learning (ML) algorithm 120 to receive the printing profile 122 and manufacture the printed product 138 according to the printing profile 122. In some examples, the additive deposition device 130 may comprise a multi-dose filament system that includes a filament palette 134 that is configured to splice and fuse a plurality of mixture filaments 132 (e.g., 132a, 132b, 132c) to form a printing load 136. In some examples, each mixture filament 132 (e.g., 132a, 132b, 132c) may include at least one polymeric matrix material and at least one active pharmaceutical ingredient (API). In the present disclosure, “polymeric matrix material,” “polymeric material,” and “polymeric matrix” are used interchangeably. The additive deposition device 130 further includes a deposition nozzle 137 that is capable of melting and depositing the printing load 136 at different nozzle angles as needed. In some examples, the additive deposition device 130 may be electrically connected to a battery 139. The battery 139 may be powered by a green or renewable energy source 137, in some examples. In some examples, the green or renewable energy source 137 may be referred as a “green energy supply.” Exemplary green or renewable energy sources include, but are not limited to, solar, wind, water, geothermal, bioenergy, or nuclear energy. In some examples, the green energy source 137 may include at least one solar panel that is portable. In some examples, the green or renewable energy source 137 may further include at least one energy monitor that measures the energy consumption of the system. In some examples, the printed product may further comprise at least one plasticizer or at least one excipient. Optionally, the at least one plasticizer is polyethylene oxide or Soluplus.

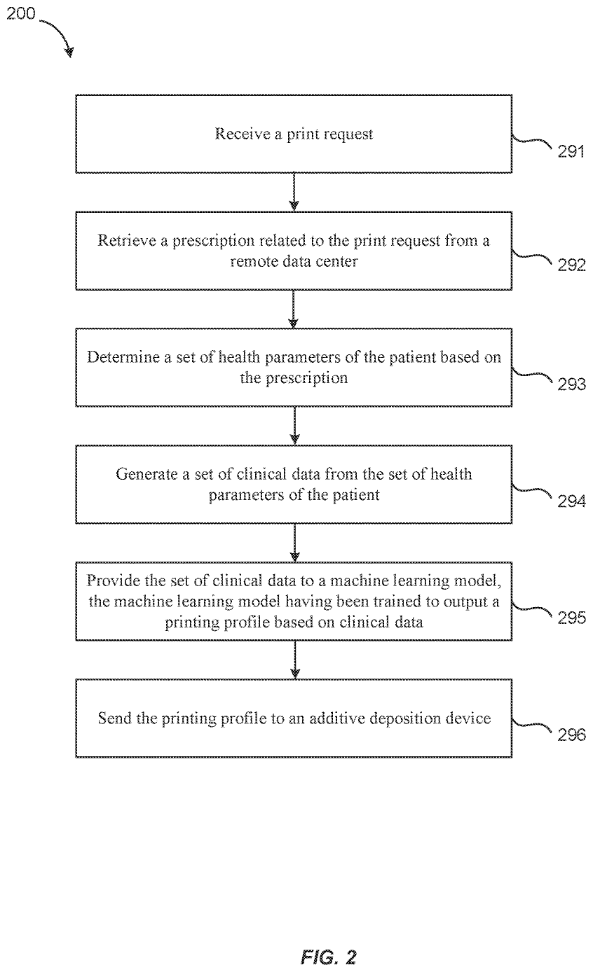

FIG. 2 is a flowchart detailing a method of manufacturing printed products according to some examples. The method 200 includes, at 291, receiving a print request. In some examples, the print request may be received from a patient's mobile communication device. The print request may be voluntarily initiated by the patient or by an initiation module that sends out the print request according to a periodic, predetermined, fixed, or variable schedule.

The method 200 further includes, at 292, retrieving a prescription related to the print request. In some examples, the prescription may be accessed from a remote data center or an external server. The prescription may include information about a dosage regimen for an active pharmaceutical ingredient.

The method 200 further includes, at 293, determining a set of health parameters of the patient based on the prescription. Referring back to FIG. 1, in some examples, the set of health parameters may be obtained from the monitoring sensor 114, voluntary input (e.g., self-reported values) from a patient, and/or accessed from an external database.

The method 200 further includes, at 294, generating a set of clinical data from the set of health parameters of the patient. In some examples, the set of clinical data may be generated by a generation module that includes at least one artificial intelligence or machine learning model.

The method 200 further includes, at 295, providing the set of clinical data to a machine learning model or artificial intelligence system, the machine learning model or artificial intelligence system having been trained to output a printing profile based on the clinical data and/or the prescription. The printing profile may include any information that is relevant to any property of a printed product. The printing profile may include, but is not limited to, filament selection information, geometry information of the printed product, splicing information that specifies the arrangement of a plurality of printing filaments for the additive deposition device 130, composition information (e.g., respective weight percentages of multiple active pharmaceutical ingredient (“API”) components and polymeric material component for manufacturing the printed product). In some examples, the printing profile may include 3D design file of the printed product as a computer-aided design (CAD) file. In some examples, the printing profile may be included in an instruction that is executable by a computing device or a manufacturing device.

The method 200 further includes, at 296, sending the printing profile to an additive deposition device for using in manufacturing a printed product, such as for manufacturing a printed product that comprises at least one polymeric material and at least one API.

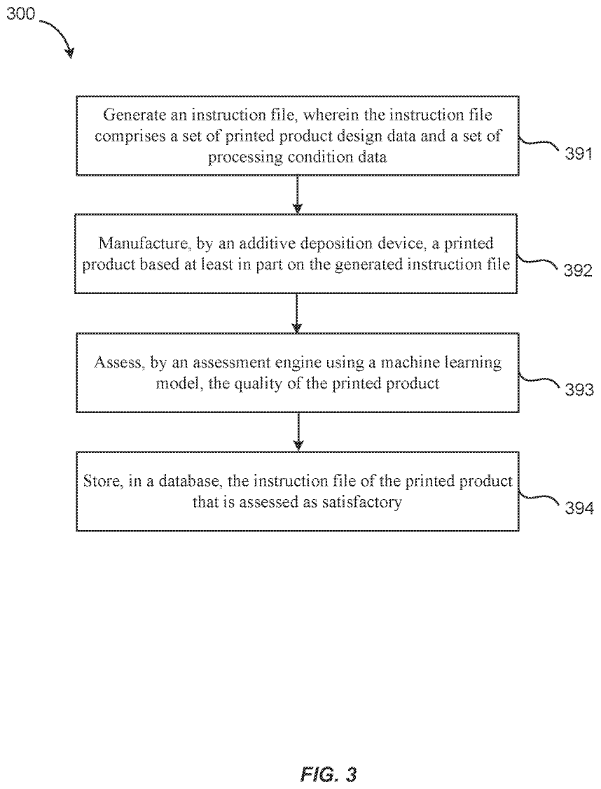

FIG. 3 is a flowchart of constructing a database according to some examples. The method 300 includes, at 391, generating an instruction file, such as an instruction file that comprises a set of printed product design data and a set of processing condition data. The set of printed product design data may specify the shape, geometry, composition information (e.g., the respective weight percentages of a polymeric and an API component), and/or any property of the printed product as desired. In some examples, the set of processing condition data may specify any processing parameters of an additive deposition device. Exemplary processing condition data may include, but is not limited to, deposition nozzle angle, nozzle temperature, nozzle type, a desired number of nozzles, deposition speed, deposition temperature, or the like. In some examples, the set of printed product design data and the set of processing condition data may be generated by a machine learning model or an artificial intelligence system, such as where an input to the machine learning model or artificial intelligence system may be a desired property of a printed product. In some examples, referring back to FIG. 1, the instruction file may contain the printing profile 122.

The method 300 further includes, at 392, manufacturing, by an additive deposition device, a printed product based at least in part on the generated instruction file. In some examples, the additive deposition device may be a 3D printer. In some examples, additional manufacturing steps that are not included in the generated instruction file may be incorporated to the manufacturing of the printed product as needed. For example, the additive deposition device may further inject a gel-based API into the printed product.

The method 300 further includes, at 393, assessing, by an assessment engine using a machine learning model, the quality of the printed product. The assessment engine may be an algorithm that is executable by a computing device. The quality of the printed product may be characterized by any property or characteristic as needed. In some examples, the quality of the printed product may be characterized by the product's weight/mass, density, mechanical strength (e.g., ultimate tensile strength or yield strength), geometry, structure, hardness, or the like. In some examples, the assessment engine may be communicatively connected to a set of quality sensors. For example, the set of quality sensors may measure and monitor any properties that are related to the performance of the printed product such as pharmaceutical tolerance or regulatory pharmaceutical values. In some examples, the quality of the printed product may be presented by a numerical value and the assessment engine may compare the quality of the printed product to a predetermined threshold value to determine if the quality of the printed product is satisfactory. For example, when density is used as the characterization property, the assessment engine can set the predetermined threshold to 1.5 g/cm³, and only printed products having a density over 1.5 g/cm³ is assessed or determined as having satisfactory quality.

The method 300 further includes, at 394, storing, in a database, the instruction file of the printed product that is assessed as satisfactory. The database may optionally be stored in a component or storage system of the additive deposition device. In some examples, the database may be stored on an external server, data center, or the like that is accessible to the additive deposition device.

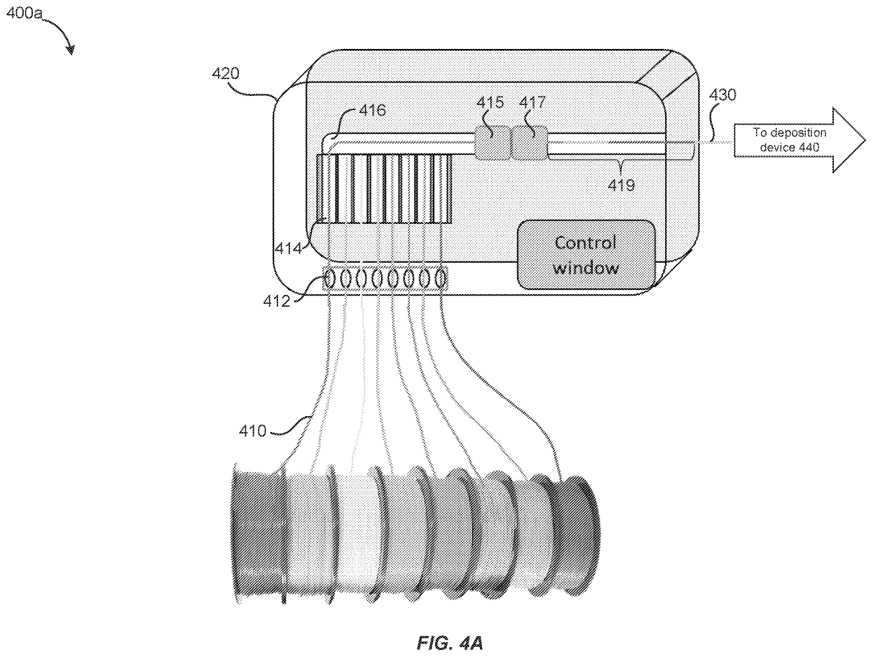

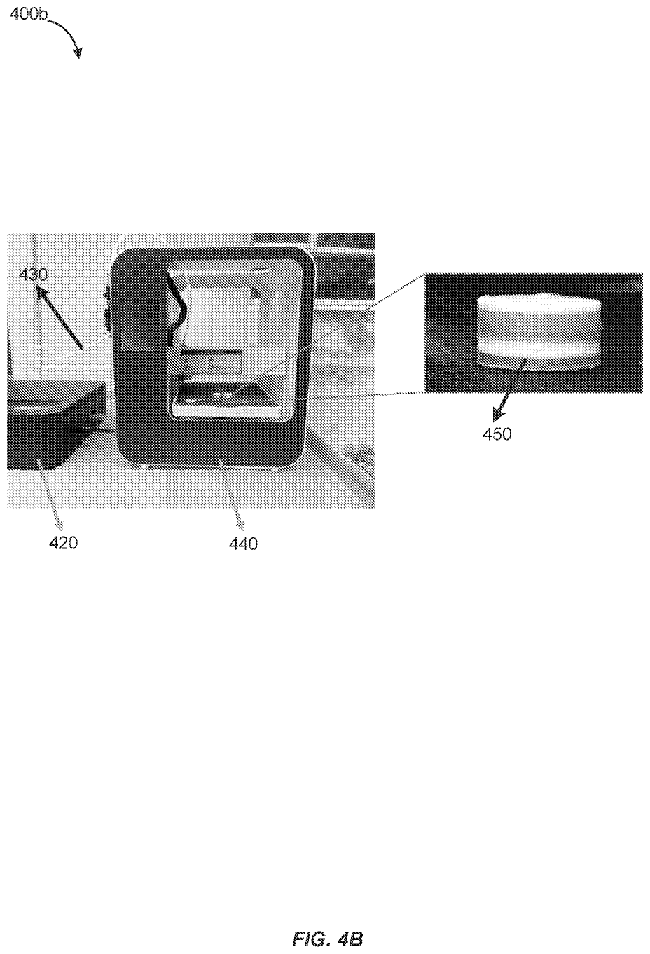

FIG. 4A shows a filament palette 400a according to some examples. The exemplary filament palette 400a, which may be the same as or different from filament palette 134 in FIG. 1, includes at least one filament 410, a main palette body 420, and a printing filament feeder 430 that is connected to an additive deposition device 440. In some examples, each filament 410 may include at least one API in a polymetric matrix. The filament 410 may have properties (e.g., consistent diameter, flexibility, strength, etc.) that are suitable to be used as 3D printing filaments. In some examples, the filament 410 may contain an API in a crystalline state, semi-crystalline state, or amorphous state. The filament 410 may be fashioned into sections of any desired lengths and may optionally be wound around a spool. In some examples, each section of a single filament 410 may contain different API and/or polymeric matrix from other sections of the same filament. In some examples, multiple filaments 410 may be loaded to the filament palette 400a, and each of the multiple filaments 410 may contain a different API and/or polymetric matrix.

The main palette body 420 selects, processes, and/or combines the at least one filament 410 to form a printing load 419 that is loaded to the additive deposition device 440 for further processing through the printing filament feeder 430. The main palette body 420 may further include at least one entry port 412 through which a filament may be loaded into the main palette body 420. The at least one entry port 412 may be connected to a separation slot 414 that is coupled to a capillary 416. The capillary 416 is equipped with processing elements. In some examples, the capillary 416 may be equipped with a cutting element 415 that sections the filament being loaded into the filament palette 400a and a heating element 417 that fuses sectioned filaments to form the printing load 419 that includes different filaments and drug combinations as desired. Referring back to FIG. 1, the printing load 419 may be equivalent to the printing load 136. In some examples, based at least in part on the printing profile 122 determined by the AI or ML algorithm 120, the filament palette 400a may instruct the main palette body 420 to cut, fuse, and/or combine multiple filaments 410 to form the printing load 419 that complies with the requirements specified in the printing profile. In some examples, the printing filament feeder 430 may include multiple outlets that are connected to the additive deposition device 440. In examples, each outlet may individually load a printing load 419 to the additive deposition device 440. In some examples, the system 100 in FIG. 1 may include any number of filament palettes. In some examples, the main palette body 420 may include a control window that displays the processing status and enables a user to monitor the process in a real-time manner.

FIG. 4B shows a configuration 400b of a filament palette 420 and an additive deposition device 440 according to some examples. As illustrated, the filament palette 420 is connected to the additive deposition device 440 to manufacture a printed product 450 based at least in part on the printing profile 122 determined by the AI or ML algorithm 120, such as described above with reference to FIG. 1. The filament palette 420 provides feeder printing load 419 via the printing filament feeder 430. As illustrated by FIG. 4B, the printed product 450 may have a multi-layer structure, such as with each layer containing a different API and/or a different polymeric matrix from at least one adjacent layer. The printed product 450 may have any shape or geometry as desired; possible shapes include, but are not limited to, cylindrical, cuboidal, caplet-like, torus-based, or film-based shapes. The printed product 450 may have any structure as desired. The printed product 450 may be or comprise amorphous or crystalline material. The API and the polymeric material or the polymeric matrix of the printed product 450 may be amorphous, semi-crystalline, or crystalline.

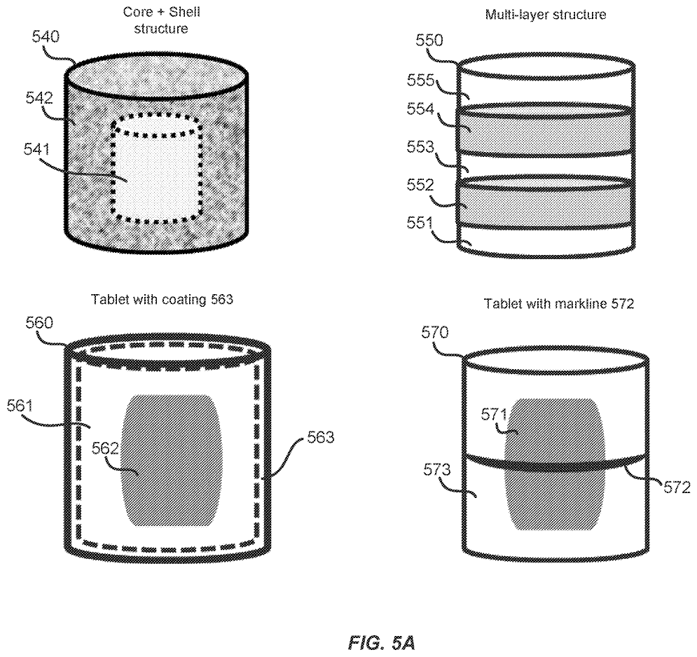

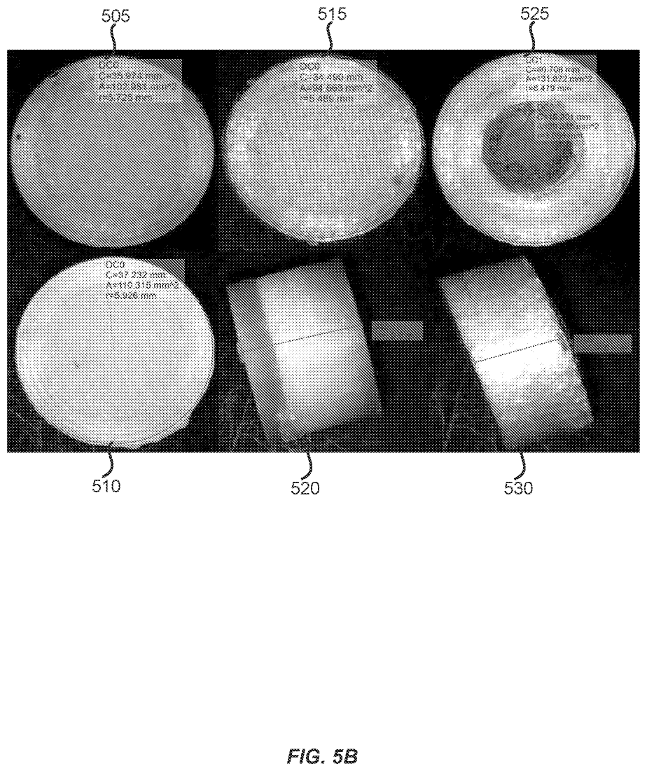

FIG. 5A is a schematic illustration of various printed product structures according to some examples. Printed product 540 comprises a core part 541 and a shell part 542 inside core part 541. In some examples, the core part 541 can be a first drug segment that further comprises a first API and a polymeric material. Additionally, the core part 541 is not limited to 3D printed drug segments and the core part 541 can be or may include a liquid-or gel-based API that is injected via a syringe or any similar device. The shell part 542 may be a compartment that houses the first drug segment to control the release of the API in the first drug segment. The shell part 542 may also be a second drug segment that further comprises a second API and/or a second polymeric material. In some cases, the shell part 542 may provide controlled release of core part 541 after some time period or under certain conditions; for example, shell part may comprise material that can break down only under certain pH conditions, such as to ensure release of core part 541 in a particular part of the digestive system. Printed product 540 is for illustration purposes only. The configuration of the core part and the shell part is not limited to any specific design. It will be appreciated that a printed product may comprise more than one core part and/or shell part.

Printed product 550 has a multi-layer structure including layer 551, layer 552, layer 553, layer 554, and layer 555. Each of layer 551, layer 552, layer 553, layer 554, and layer 555 may individually be a drug segment that further comprises at least one API and/or a polymeric material, a polymeric material layer, an API layer, a coating layer such as a sugar coating layer to disguise the taste of the API, a release control coating layer to delay the release of the API, or any substance or materials as desired. Additionally, each layer (e.g., layer 551, layer 552, layer 553, layer 554, and layer 555) is not required to be 3D printed, and each layer can be or may further include liquid or gel that is injected via a syringe or any similar device. It will be appreciated that the number, the shape or geometry, the arrangement, and the sequence of the layers are not limited to the structure described as printed product 550.

Printed product 560 comprises a core part 562, a shell part 561, and a coating part 563 encapsulating the shell part 561. Each of the core part 562, the shell part 561, and the coating part 563 may optionally be a drug segment that comprises at least one API and/or a polymeric material, a polymeric material layer, an API layer, or any substance or materials as desired. The coating part 563 may be any coating layer as desired. Exemplary coating part 563 includes a sugar coating layer to disguise the taste of the API, a release control coating layer to delay the release of the API, or the like.

Printed product 570 comprises a core part 571, a shell part 573, and a markline 572 visibly embedded on the surface of the shell part 573. The markline 572 may be deposited as a very thin layer that forms a slice of the shell part 573. In some embodiments, the markline 572 may also be deposited directly on the surface of the shell part 573 by using 3D printing or any similar depositing technology. Printed product 570 may include more than one markline and the marklines may be arranged in any pattern for aesthetic, marking, or any purposes as desired. In some examples, the markline 572 may correspond to a recessed region in printed product 570. Optionally, the markline 572 may facilitate cutting or breaking the printed product 570.

Although printed products 540, 550, 560, and 570 depict printed products in cylindrical tablets or elongated tablets, the shape or geometry of a printed product is not limited to the examples depicted, and irregular or complex shapes, such as donut shape, star shape, heart shape, or the like, can be used. For example, the techniques describe herein may also be used to make different printed products including APIs in any desirable form or shape, such as thin-films, microneedles, etc.

FIG. 5B shows printed products according to some examples. FIG. 5B shows top views of a first exemplary printed product 505 and a second exemplary printed product 510 with uniformly solid structures. FIG. 5B also shows a top view of a third exemplary printed product 515 and side view of a fourth exemplary printed product 520 with multi-layer structures. FIG. 5B also shows a top view of a fifth exemplary printed product 525 and a side view of a sixth exemplary printed product 530 with concentric structures.

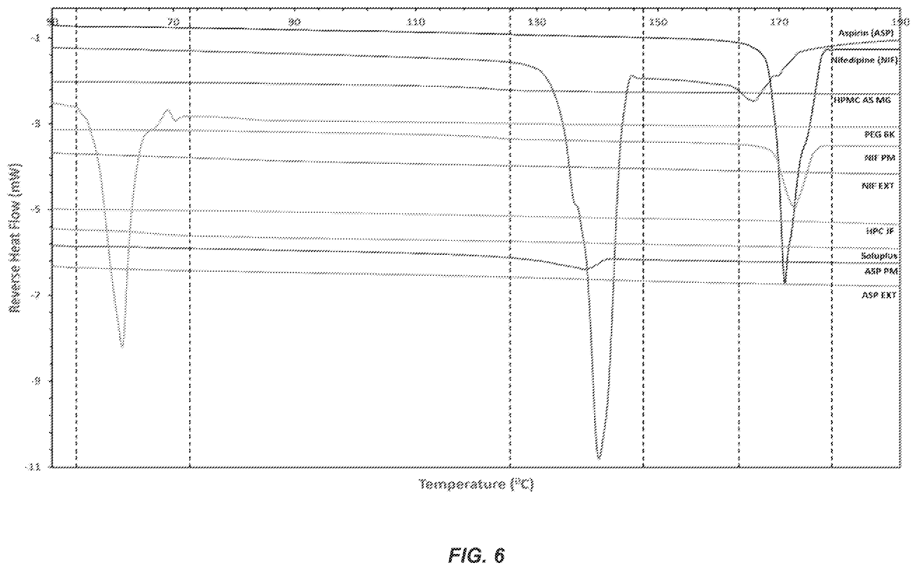

FIG. 6 provides data showing differential scanning calorimetry analysis results of pharmaceutical ingredients, filaments, and printed products according to some examples. In some cases, analysis by differential scanning calorimetry can confirm components and/or amounts of components in a printed product.

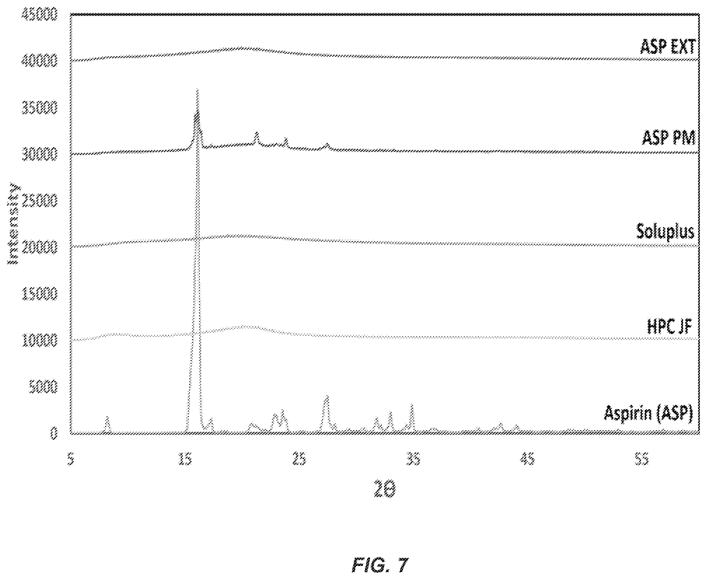

FIG. 7 provides data showing powder X-ray diffraction analysis results of pharmaceutical ingredients, filaments, and printed products according to some examples.

The results show that the processing conditions are maintained such that API in the printed product is completely rendered amorphous after the process. In some cases, analysis by powder X-ray diffraction can confirm components and/or amounts of components in a printed product.

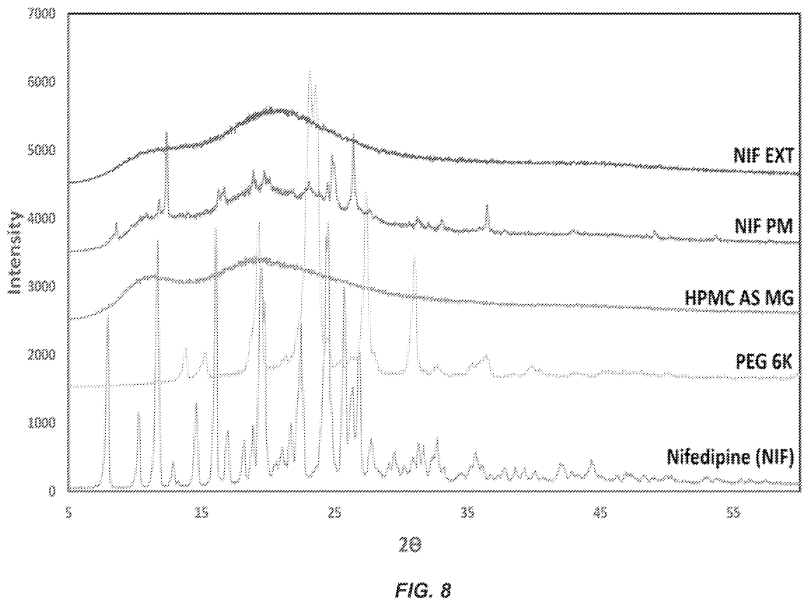

FIG. 8 provides data showing powder X-ray diffraction analysis results of pharmaceutical ingredients, filaments, and printed products according to some examples. The results show that the processing conditions are maintained such that API in the printed product is completely rendered amorphous after the process. In some cases, analysis by powder X-ray diffraction can confirm components and/or amounts of components in a printed product.

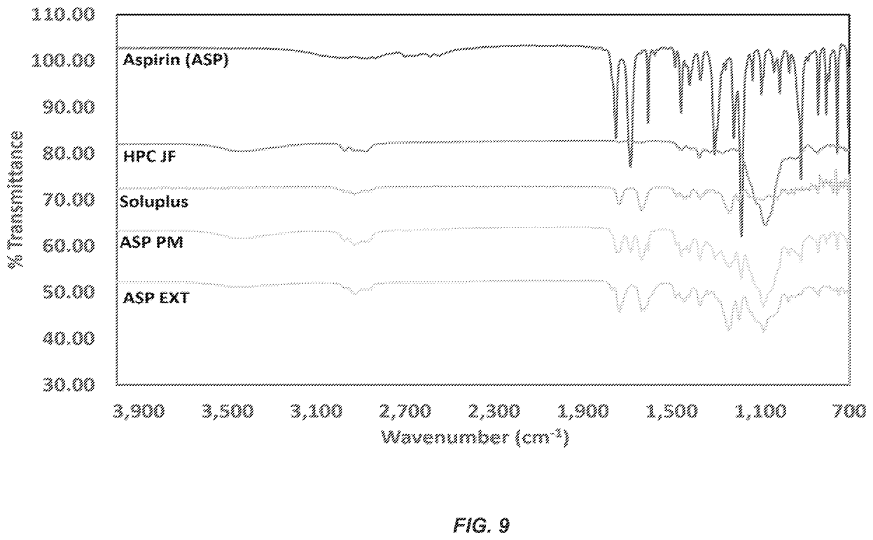

FIG. 9 provides data showing Fourier-transform infrared spectroscopy analysis of pharmaceutical ingredients, filaments, and printed products according to some examples. In some cases, analysis by Fourier-transform infrared spectroscopy can confirm components, amounts of components, and/or drug-excipient interactions in a printed product.

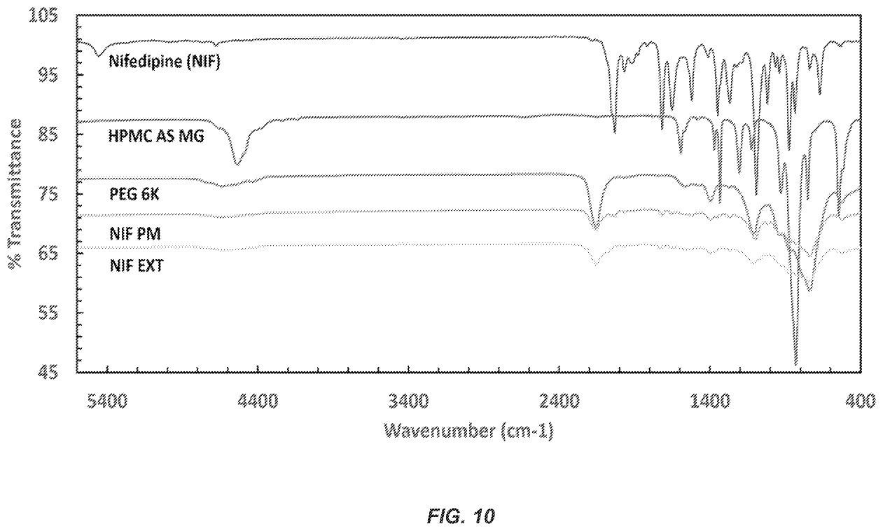

FIG. 10 provides data showing Fourier-transform infrared spectroscopy analysis results of pharmaceutical ingredients, filaments, and printed products according to some examples. In some cases, analysis by Fourier-transform infrared spectroscopy can confirm components, amounts of components, and/or drug-excipient interactions in a printed product.

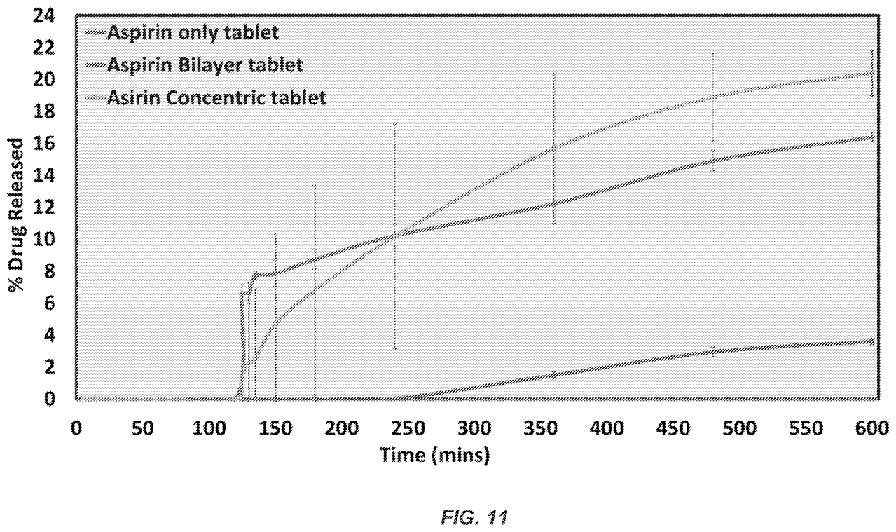

FIG. 11 provides data showing drug release profiles of an exemplary pharmaceutical ingredient according to some examples. In some cases, printed products having different structures have different drug release profiles.

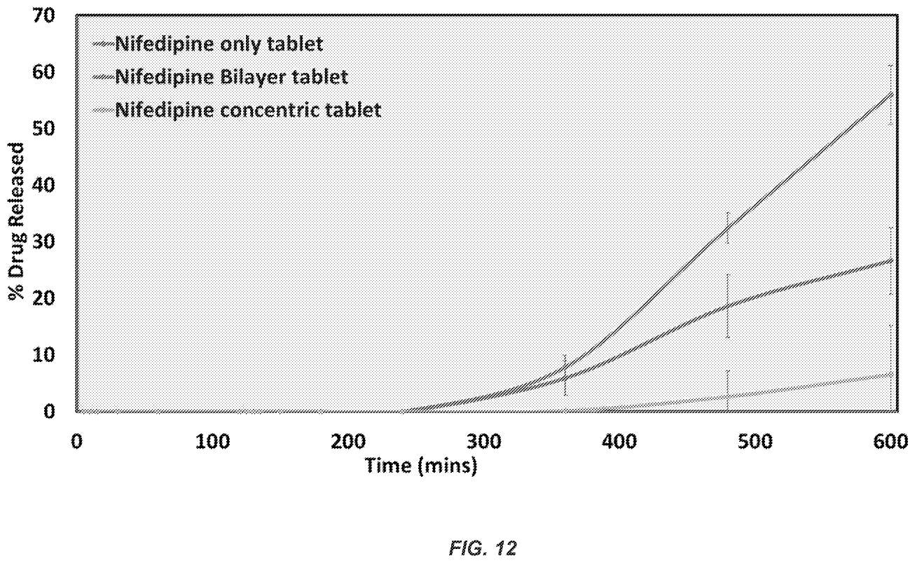

FIG. 12 provides data showing drug release profiles of an exemplary pharmaceutical ingredients according to some examples. In some cases, printed products having different structures have different drug release profiles.

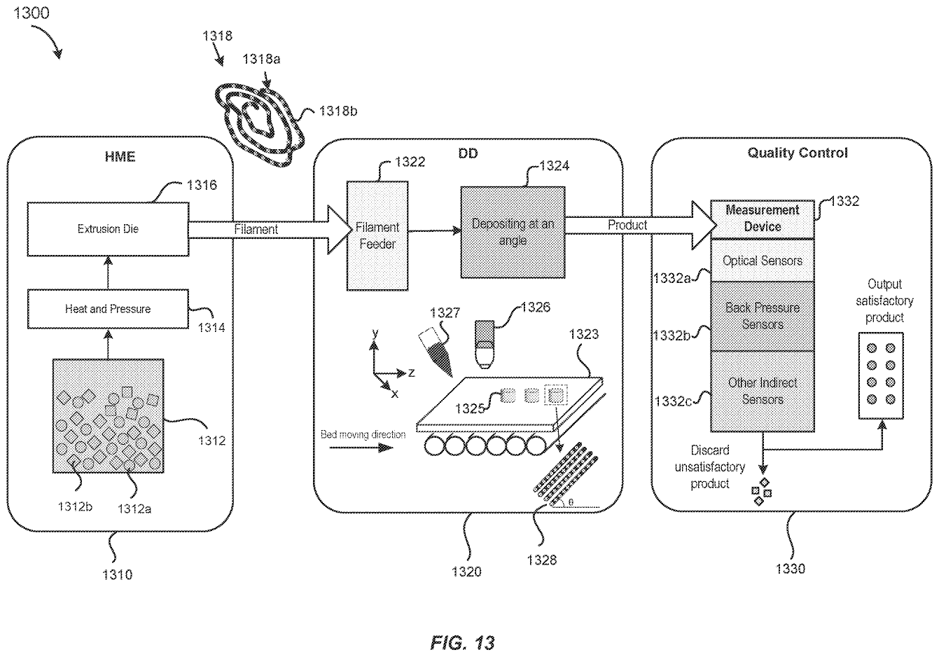

FIG. 13 is a schematic illustration of manufacturing filaments according to some examples. The system 100, method 200, and method 300 may be implemented as part of or in combination with system 1300. The system 1300 includes a hot melt extruder (HME) 1310, a deposition device (DD) 1320, and a quality control component 1330. As illustrated, API 1312a and polymeric matrix material 1312b are physically mixed to form a mixture 1312 that can be fed into the HME 1310. Within the HME barrel, the mixture 1312 is heated and pressurized at a processing chamber 1314. The processing chamber 1314 may include any equipment or device as needed. In some examples, the processing chamber 1314 may include screws and/or heaters. The mixture 1312 is then forced through the extrusion die 1316 to form an extruded filament 1318. In some examples, within the HME, the processing conditions (e.g., temperature, screw configuration, feed rate, screw speed, etc.) are maintained until the polymeric matrix material 1312b and optionally the API 1312a are molten and/or the API 1312a solubilizes or is suspended or mixed in the polymeric matrix 1312b. After cooling or any other optional processing steps, the processed mixture 1312 exiting the die 1316 forms extruded filament 1318 comprising the API 1318a in the polymetric matrix 1318b. In examples, the extruded filament 1318 has properties (e.g., consistent diameter, flexibility, strength, etc.) suitable to be used as 3D printing filaments. The extruded filament 1318 may contain the API in crystalline state, semi-crystalline state, or amorphous state. The extruded filament 1318 may be fashioned into sections of any desired lengths and may optionally be wound around a spool.

In some examples, the extruded filament 1318 can be fed to a deposition device 1320 via a filament feeder 1322. The deposition device 1320 may be continuously connected with the HME 1310 to form an integrated processing line for large-scale manufacturing, but this is not required in all examples, and filament 1318 can be manually provided (e.g., as a spool or lengths of filament 1318) to or as part of filament feeder 1322. In some examples, the deposition device 1320 involves the additive deposition of molten feedstock or filament extruded through a computer-controlled deposition nozzle 1326. The deposition device 1320 can be capable of creating complex geometries as well as 3D models with controlled composition and architecture. In some examples, the deposition device 1320 may comprise a hot-end part 1324 that includes the computer-controlled deposition nozzle 1326 and a relatively-cooler-end part that includes a build platform 1323. To build a printed product 1325, the deposition device 1320 injects the molten filaments in a layer-by-layer fashion according to the structure and geometry of the printed product 1325 while controlling position of the deposition nozzle 1326 and build platform 1323. The printed product 1325 may have any shape or geometry as desired; possible shapes include, but are not limited to, cylindrical, cuboidal, caplet-like, torus-based, or film-based shapes.

In some examples, when the extruded filament 1318 enters the hot end part 1324, the extruded filament 1318 is heated to its transition temperature. As the extruded filament 1318 becomes softened or molten, the viscosity of the filament is reduced. The molten filament 1318 is then extruded through the computer-controlled deposition nozzle 1326 onto the build platform 1323. The computer-controlled deposition nozzle 1326 may deposit the molten filament at different nozzle angles, which can provide for an unlimited dimension for continuous printing, such as where the build platform 1323 is a conveyor belt. The nozzle angle can be changed according to processing needs. In some embodiments, the nozzle angle is selected to be 45° to avoid excessive building of support layers. Printed product 1328 shows an exemplary printed product built with a nozzle angle (θ) of 45°. Furthermore, to diversify the materials that can be used, an extrusion syringe 1327 along with the nozzle head may be incorporated to the deposition device 1320. The extrusion syringe 1327 can be a semi-solid extrusion syringe that is capable of printing using gel or liquid-based materials that may be susceptible to thermal degradation. The extrusion syringe 1327 may be actuated via a mechanical pump or any pressure-assisted mechanism, for example. Besides the extrusion syringe discussed above, any alternative kind of liquid dispenser may be used. The printed product 1328 may be amorphous or crystalline. The API and the polymeric material of the printed product may be amorphous, semi-crystalline, or crystalline.

In some examples, the build platform 1323 may be a dynamic platform such as a conveyor belt that moves toward the z-axis direction as the printing continues, or any similar configurations. The x-y plane defines the surface of the build platform 1323. Although FIG. 13 illustrates a dynamic build platform 1323 that moves unidirectionally along the z-axis, the dynamic build platform 1323 may also move or shift the build platform toward more than one direction (e.g., toward the x-axis, the y-axis, or a combination of alternate movements toward the x-axis and the y-axis, etc.) when desired. Furthermore, as the computer-controlled deposition nozzle 1326 may move along the x-axis, y-axis, or z-axis direction, the printed product 1325 may be deposited at any location on the surface of the build platform 1323, and the layout of the printed product 1325 on the platform 1323 is not limited to any specific arrangement of rows or columns.

In some examples, a quality control component 1330 may be integrated as an in-line monitoring block for optional downstream processing. Various characteristics, factors, or values of the printed product 1325 may be monitored to ensure the product quality, reproducibility, and identify possible API degradation. An exemplary quality control block 1330 may include optical sensors 1332a that measure and interpret the electromagnetic spectra that result from the interaction between electromagnetic radiation and the printed product 1325 as a function of the wavelength or frequency of the radiation. Exemplary optical sensors 1332a include infrared (IR) spectroscopy, ultraviolet-visible-near-IR Spectroscopy (UV-Vis-NIR), Fourier transform infrared spectroscopy (FTIR), or the like. The optical sensors 1332a may also include optical spectrometers (e.g., spectrophotometer, spectrograph, or spectroscope) that measure properties of light over a specific portion of the electromagnetic spectrum to identify materials and/or properties. In some embodiments, the optical sensors may be NIR fiber optic probes or the like.

In some examples, the quality control component 1330 may further include back pressure sensors 1332b to measure and monitor the force or pressure of molten filaments or fluids within the computer-controlled deposition nozzle 1326 or the extrusion syringe 1327 to ensure that the deposition is progressing properly.

In some examples, the quality control component 1330 may also include other indirect sensors 1332c to measure various properties of the printed product of the printed product 1325 and to monitor each stage of the manufacturing process. Properties may be measured and monitored include mass, density, material structure, and any other properties related to pharmaceutical tolerance or regulatory pharmaceutical values. The quality control component 1330 can separate satisfactory printed product from unsatisfactory printed product according to various quality control factors. Satisfactory printed product can be output to the following processing stages like packing (not shown) while unsatisfactory product may be discarded or optionally recycled to HME 1310.

### REFERENCES

The following references, to the extent that they provide exemplary procedural or other details supplementary to those set forth herein, are hereby incorporated by reference.
Zheng et al., Melt extrusion deposition (MED™) 3D printing technology—A paradigm shift in design and development of modified release drug products, International Journal of Pharmaceutics, Volume 602, 2021, 120639, ISSN 0378-5173.
U.S. Pat. No. 11,364,674

### STATEMENTS REGARDING INCORPORATION BY REFERENCE AND VARIATIONS

All references throughout this application, for example patent documents, including issued or granted patents or equivalents and patent application publications, and non-patent literature documents or other source material are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “1, 2, 3, 1 and 2, 1 and 3, 2 and 3, or 1, 2, and 3”.

Every formulation or combination of components described or exemplified can be used to practice the invention, unless otherwise stated. Specific names of materials are intended to be exemplary, as it is known that one of ordinary skill in the art can name the same material differently. It will be appreciated that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The invention illustratively described herein suitably may be practiced in the absence of any element, elements, limitation, or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A system comprising:
a computing device that is configured to send an instruction, wherein the instruction includes a set of composition data and a set of structure data of a printed product;
a multi-dose filament system that is configured to receive the instruction from the computing device and to produce a mixture filament including at least one polymeric material and at least one active pharmaceutical ingredient (API) according to the set of composition data received from the computing device; and
an additive deposition device that is configured to receive the instruction from the computing device and to manufacture the printed product according to the set of structure data by using a printing load that includes the mixture filament.

2. The system of claim 1, wherein the system is powered by a green energy supply.

3. (canceled)

4. The system of claim 1, further comprising at least one energy monitor that measures energy consumption of the system, or further comprising at least one in-line monitor that continuously measures a characteristic of the printed product by using back pressure sensors or optical sensors, or further comprising at least one filament palette configured to splice and fuse a plurality of mixture filaments to form the printing load.

5. (canceled)

6. (canceled)

7. The system of claim 1 wherein the multi-dose filament system is configured to produce the mixture filament by using a plurality of different polymeric materials and/or a plurality of different APIs.

8. A method comprising:
receiving a print request;
retrieving a prescription related to the print request;
determining a set of health parameters of a patient based on the prescription;
generating a set of clinical data from the set of health parameters of the patient;
providing the set of clinical data to a machine learning model, the machine learning model having been trained to output a printing profile based on clinical data; and
sending the printing profile to an additive deposition device for using in manufacturing a printed product, wherein the printed product comprises at least one polymeric material and at least one API.

9. The method of claim 8 wherein the print request is received from a patient's mobile communication device.

10. The method of claim 8 wherein the prescription related to the print request is retrieved from a remote data center.

11. (canceled)

12. (canceled)

13. The method of claim 8, wherein the printing profile comprises filament selection information and geometry information of the printed product, or wherein the printing profile comprises splicing information that specifies selection of a plurality of printing filaments for the additive deposition device, or wherein the printing profile comprises composition information that specifies the respective weight percent of the at least one API and the at least one polymeric material for manufacturing the printed product.

14. (canceled)

15. (canceled)

16. (canceled)

17. The method of claim 8, further comprising receiving an approval of the print request from a healthcare provider before sending the printing profile to the additive deposition device.

18. The method of claim 8, further comprising manufacturing a printed product based on the printing profile via an additive deposition device.

19. A system comprising:
one or more processors; and
one or more memory storing instructions that, upon execution by the one or more processors, configure the system to:
receiving a print request;
retrieving a prescription related to the print request;
determining a set of health parameters of a patient based on the prescription;
generating a set of clinical data from the set of health parameters of the patient;
providing the set of clinical data to a machine learning model, the machine learning model having been trained to output a printing profile based on clinical data; and
sending the printing profile to an additive deposition device for using in manufacturing a printed product, wherein the printed product comprises at least one polymeric material and at least one API.

20. The system of claim 19, wherein the print request is received from a patient's mobile communication device.

21. The system of claim 19, wherein the prescription related to the print request is retrieved from a remote data center.

22. (canceled)

23. (canceled)

24. The system of claim 19, wherein the printing profile comprises filament selection information and geometry information of the printed product, or wherein the printing profile comprises splicing information that specifies the selection of a plurality of printing filaments for the additive deposition device, or wherein the printing profile comprises composition information that specifies the respective weight percent of the at least one API and the at least one polymeric material for manufacturing the printed product.

25. The system of claim 24, wherein the geometry information comprises a STL file that is supported by a Computer-Aided Design (CAD) software.

26. The system of claim 19, further comprising receiving an approval of the print request from a healthcare provider before sending the printing profile to the additive deposition device.

27. The system of claim 19, further comprising manufacturing a printed product based on the printing profile via an additive deposition device.

28. (canceled)

29. (canceled)

30. A method comprising:
generating an instruction file, wherein the instruction file comprises a set of printed product design data and a set of processing condition data;
manufacturing, by an additive deposition device, a printed product based at least in part on the generated instruction file;
assessing, by an assessment engine using a machine learning model, the quality of the printed product; and
storing, in a database, the instruction file of the printed product that is assessed as satisfactory.

31. The method of claim 30, wherein the set of printed product design data includes geometry of the printed product.

32. The method of claim 30, wherein the set of processing condition data includes nozzle temperature, nozzle type, or number of nozzle of the additive deposition device.
